(19) United States
(12) Patent Application Publication
(10) Pub. No.: US 2024/0079102 A1
(43) Pub. Date: Mar. 7, 2024

(54) METHODS AND SYSTEMS FOR PATIENT INFORMATION SUMMARIES

(71) Applicant: GE Precision Healthcare LLC, Wauwatosa, WI (US)

(72) Inventors: Shivappa Goravar, Bengaluru (IN); Akshit Achara, Bengaluru (IN); Sanand Sasidharan, Bengaluru (IN); Anuradha Kanamarlapudi, Bengaluru (IN)

(21) Appl. No.: 17/929,217

(22) Filed: Sep. 1, 2022

(51) Int. Cl. G16H 10/60; G06F 40/166; G06F 40/253; G06F 40/279; G06F 40/40

(52) U.S. Cl. CPC G16H 10/60; G06F 40/166; G06F 40/253; G06F 40/279; G06F 40/40

(57) ABSTRACT

Various methods and systems are provided for generating and displaying summaries of patient information extracted from one or more medical reports stored in an electronic medical record (EMR) of a patient. In one example, a method includes receiving text data of a patient; entering the text data as input into a plurality of entity recognition models, each entity recognition model of the plurality of entity recognition models trained to label instances of a respective entity in the text data; aggregating the labeled text data outputted by each entity recognition model; generating a summary of the text data based on the aggregated labeled text data; and displaying and/or saving the summary and/or the aggregated labeled text data.

### FIELD

Embodiments of the subject matter disclosed herein relate to patient information, and more particularly to automatically identifying and summarizing relevant patient information.

### BACKGROUND

Digital collection, processing, storage, and retrieval of patient medical records may include a conglomeration of large quantities of data. In some examples, the data may include numerous medical procedures and records generated during investigations of the patient, including a variety of examinations, such as blood tests, urine tests, pathology reports, image-based scans, etc. Duration of the diagnosis of a medical condition of a subject followed by treatment may be spread over time from few days to few months or even years in the case of chronic diseases, which may be diseases that take more than one year to cure. Over the course of diagnosing and treating chronic disease, the patient may undergo many different treatments and procedures, and/or may move to different hospitals and/or geographic locations.

Physicians are increasingly relying on Electronic Medical Record (EMR) systems to go through historical health records of the patient during diagnosis, treatment, and monitoring of a patient condition. For patients with chronic illnesses, there are often hundreds or even thousands of EMRs resulting from numerous visits. Sorting and extracting information from past EMRs for such patients is a slow and inefficient process, increasing a likelihood of missing records with relevant data which may be spread out across a large number of less informative routine visit records.

### BRIEF DESCRIPTION

In one embodiment, a method comprises receiving text data of a patient; entering the text data as input into a plurality of entity recognition models, each entity recognition model of the plurality of entity recognition models trained to label instances of a respective entity in the text data; aggregating the labeled text data outputted by each entity recognition model; generating a summary of the text data based on the aggregated labeled text data; and displaying and/or saving the summary and/or the aggregated labeled text data.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 illustrates a system for summarizing and displaying clinical information of a patient to a user in accordance with an aspect of the disclosure;

FIG. 2A shows a block diagram schematically illustrating a training system for training a plurality of models to recognize entities in text data, according to an embodiment of the disclosure;

FIG. 2B shows a block diagram schematically illustrating a flow of data when generating patient information summaries using a plurality of trained models, according to an embodiment of the disclosure;

FIG. 3 shows a flowchart illustrating an exemplary method for training a plurality of models to recognize entities in text data, according to an embodiment of the disclosure;

FIG. 4 shows a flowchart illustrating a high-level method for generating patient information summaries using a plurality of trained models, according to an embodiment of the disclosure;

FIG. 5 shows a flowchart illustrating an exemplary method for labeling entities in text data based on an output of a plurality of entity recognition models, according to an embodiment of the disclosure;

FIG. 6 shows a flowchart illustrating an exemplary method for assigning a label to an instance of an entity based on a relative weighting an output of a plurality of entity recognition models, according to an embodiment of the disclosure;

FIG. 7 shows a flowchart illustrating an exemplary method for resolving entity labeling conflicts in an output of a plurality of entity recognition models, according to an embodiment of the disclosure;

FIG. 8A is a first excerpt of an example output of a system for summarizing clinical information of a patient, according to an embodiment of the disclosure;

FIG. 8B is an example display of the example output of FIG. 8A, according to an embodiment of the disclosure;

FIG. 9A is a second excerpt of an example output of a system for summarizing clinical information of a patient, according to an embodiment of the disclosure;

FIG. 9B is an example display of the example output of FIG. 9A, according to an embodiment of the disclosure;

FIG. 10 is a third excerpt of an example output of a system for summarizing and displaying clinical information of a patient, according to an embodiment of the disclosure; and

FIG. 11 is a schematic diagram of a database table, according to an embodiment of the disclosure.

### DETAILED DESCRIPTION

The following description relates to various embodiments of methods and systems to summarize information within an electronic medical record (EMR) of a patient, by detecting entities of interest that are important to doctors in digitized medical reports of the EMR, and generating a summary of patient information relating to the entities of interest. The summary may be formatted in various ways and may be customized, depending on an implementation. By generating the patient summary, an amount of time spent by a caregiver reviewing medical reports included in the EMR may be reduced, thereby freeing the caregiver up to address other tasks. Additionally, an amount of relevant patient information made available to the caregiver during the caregiver's limited time spent reviewing the medical reports may be increased, resulting in improved patient outcomes.

An entity of interest (also referred to herein as an entity) may be a classification, categorization, or label associated with a text expression (e.g., a word or combination of words) found in a medical report included in an EMR. For example, “disease” may be a first entity of interest, where a first entity recognition model may be trained to label instances of words or multi-word text expressions in the medical report referring to diseases (e.g., cancer, hepatitis, coronavirus, etc.). “Anatomy” may be a second entity of interest, where a second entity recognition model may be trained to label instances of words or multi-word text expressions in the medical report referring to parts of an anatomy of a patient (e.g., heart, lung, brain, etc.). Various entities of interest may be defined, for example, by a doctor, a group of doctors, a medical association, hospital administrators, or other healthcare professionals. In some embodiments, the entities of interest may be organized in a hierarchical manner with categories and sub-categories. For example, “disease” may be a first entity of interest, which may include a category “cancer” as a second entity of interest; the category “cancer” may include a sub-category “lung cancer” as a third entity of interest; and so on. The entities of interest may be predefined, and/or may be periodically added to or changed. For example, a new category or sub-category of an entity may be added.

The entities of interest in the medical reports included in EMR may be detected using a plurality of entity recognition models, and aggregating the results from the plurality of entity recognition models. A single model trained with a single corpus of data may not perform well due to scarcity of labelled data and skewness of labelled entities. Hence, the approach disclosed herein involves a suite of models that may be developed depending the quantity of labelled data and categories of entities, and selecting a suitable list of models for generating a summary of an EMR based on a specific scenario/specific dataset of the EMR. Various steps in the proposed approach may include identifying/collecting labelled/annotated dataset(s); identifying entities of interest to doctors and/or other clinicians; training each entity recognition model specific to a single entity or multiple entities; selecting a group of trained models suitable for use during inference for a specific scenario or type of dataset; predicting the entities of interest from the selected group of trained models; aggregating the output from the plurality of models and resolving any labeling conflicts; and using prior information of model performance/rules derived from domain knowledge to refine the output.

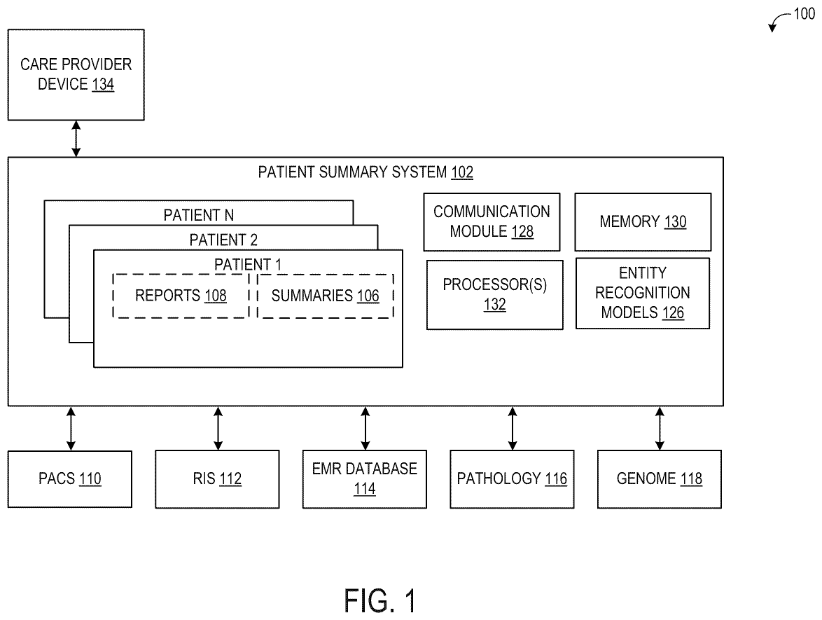

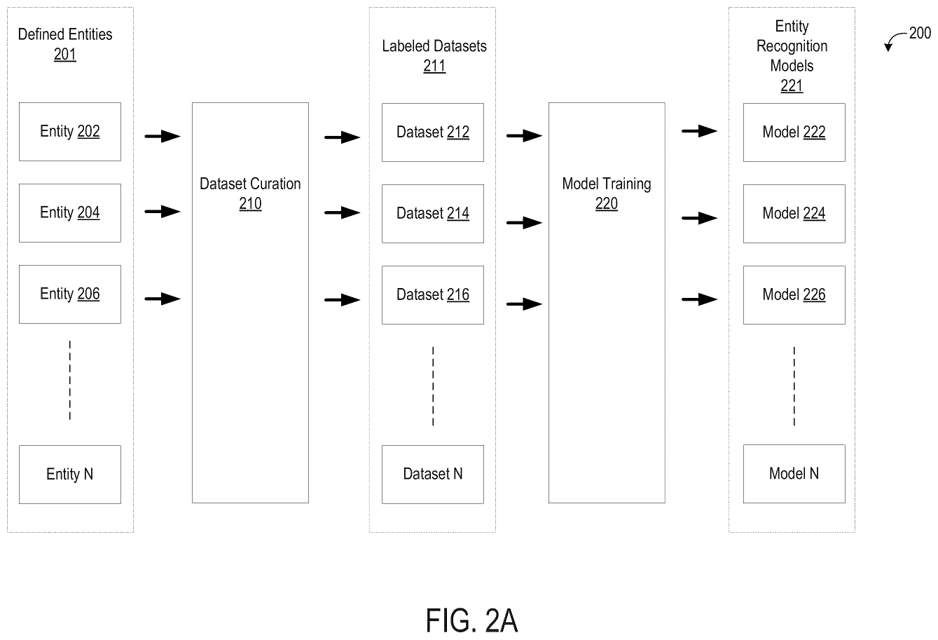

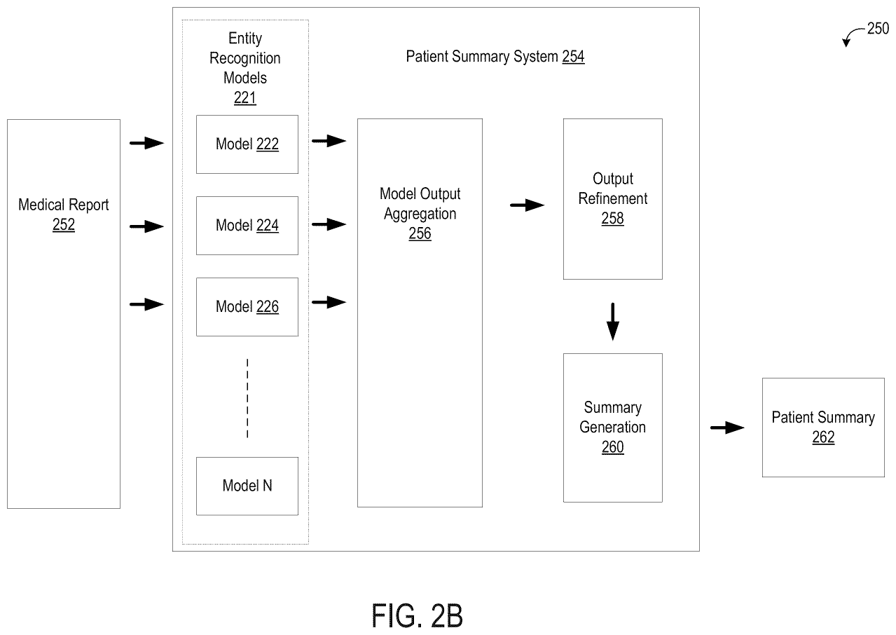

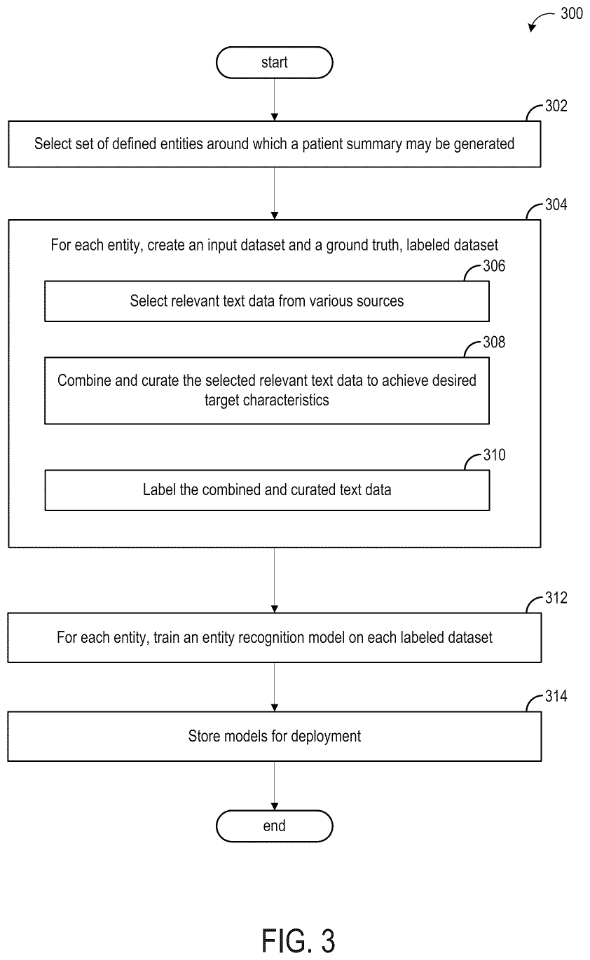

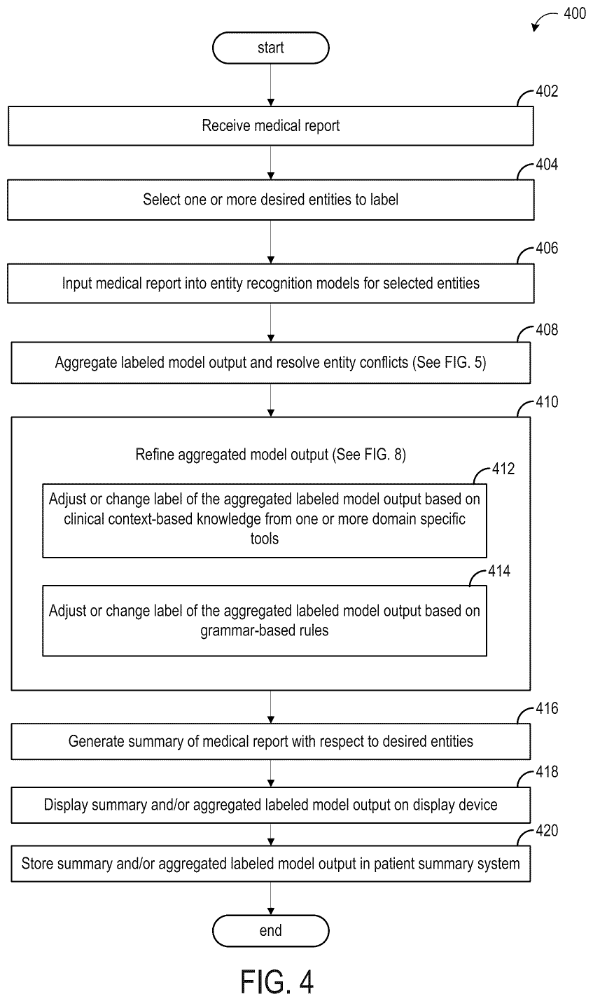

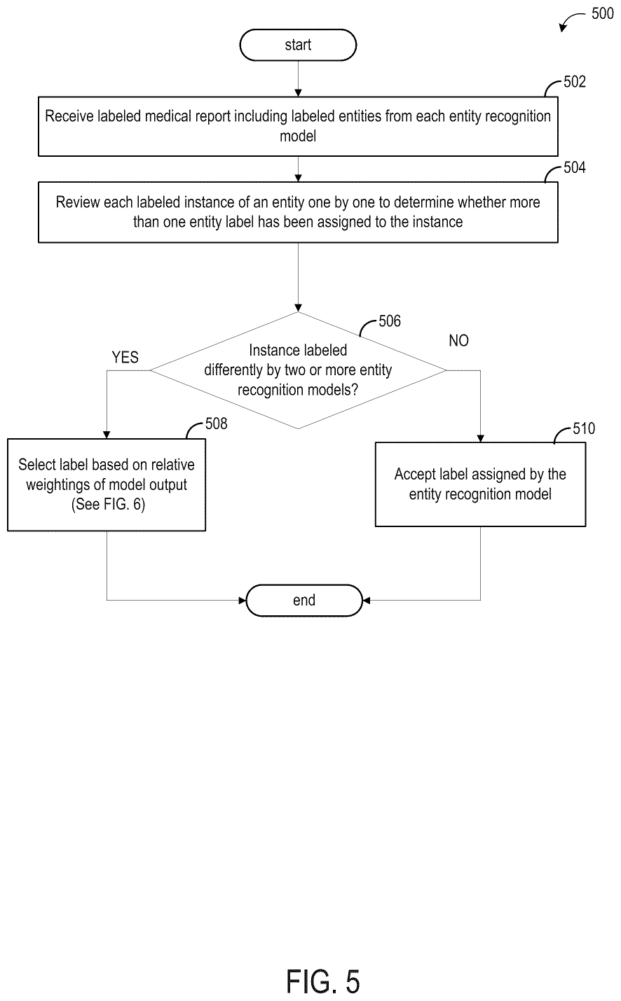

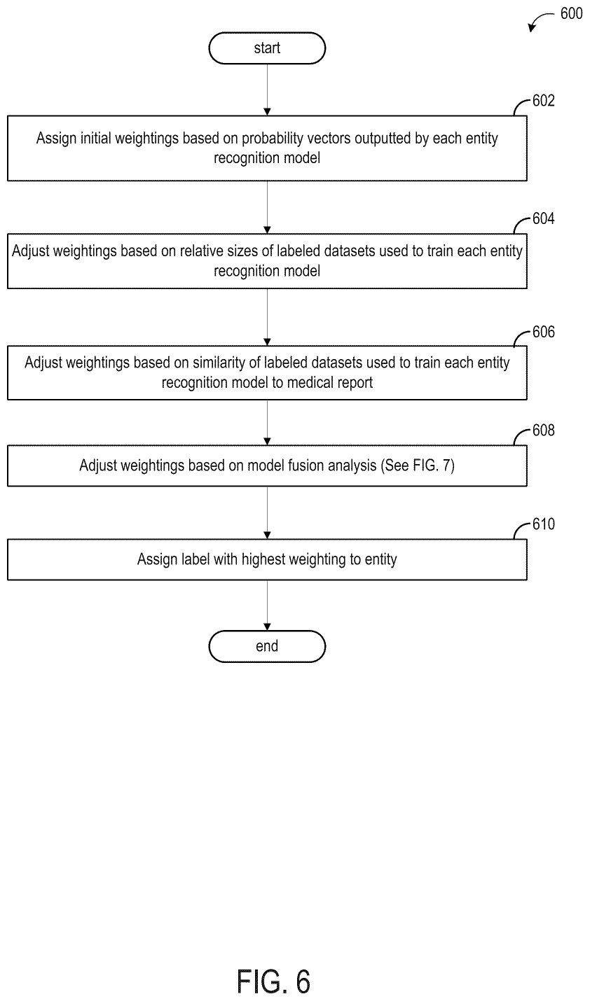

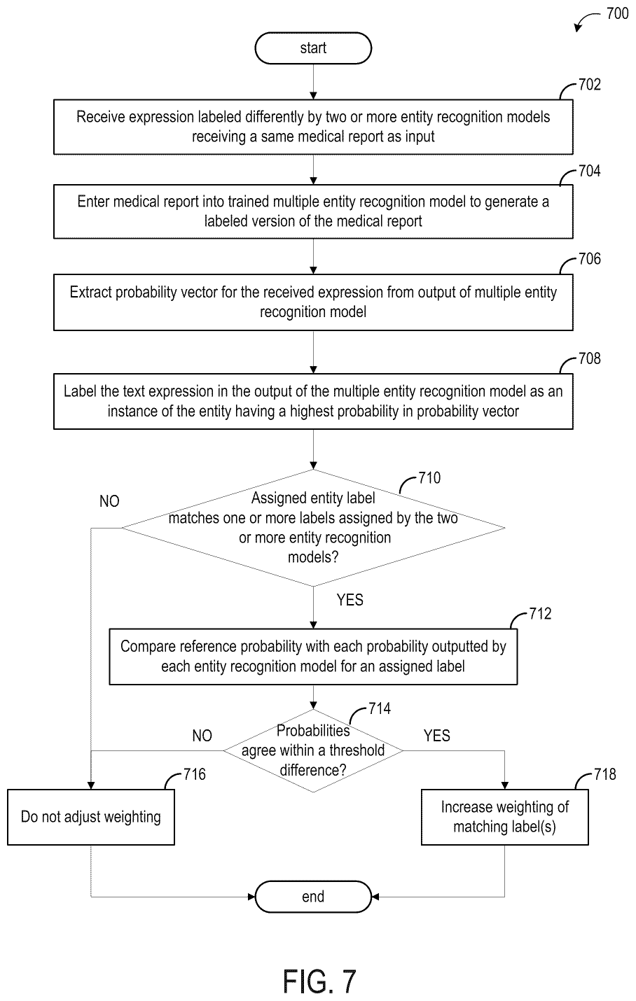

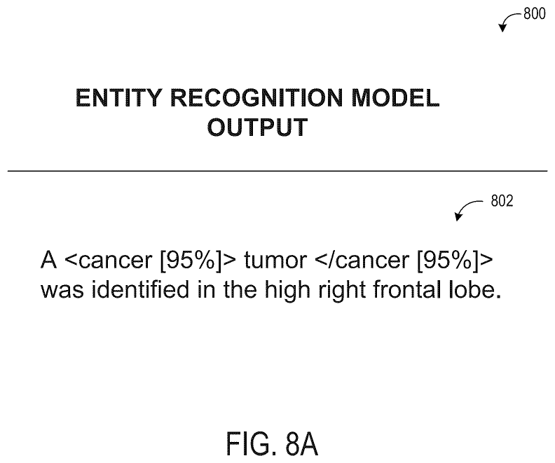

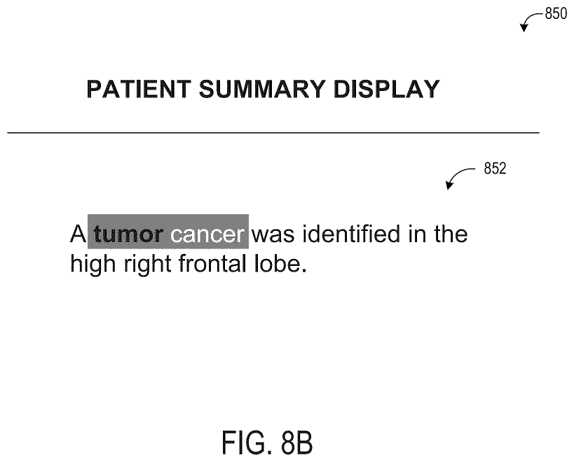

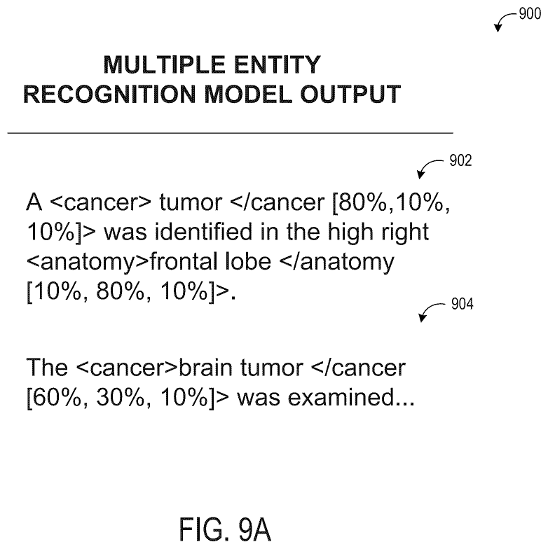

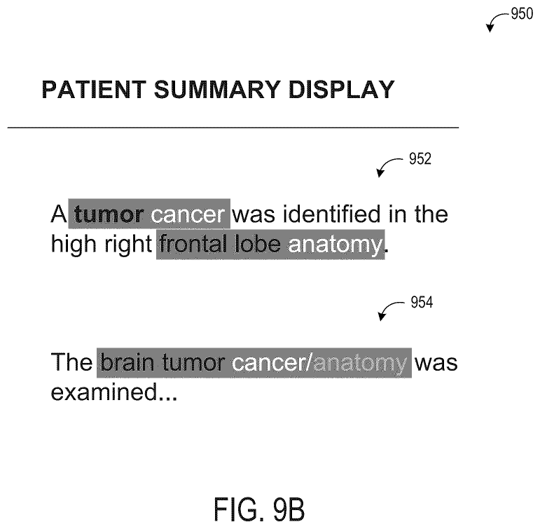

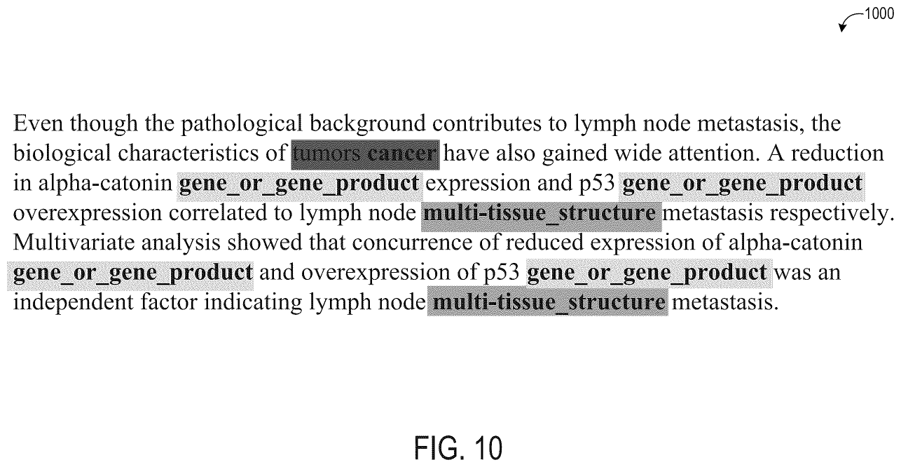

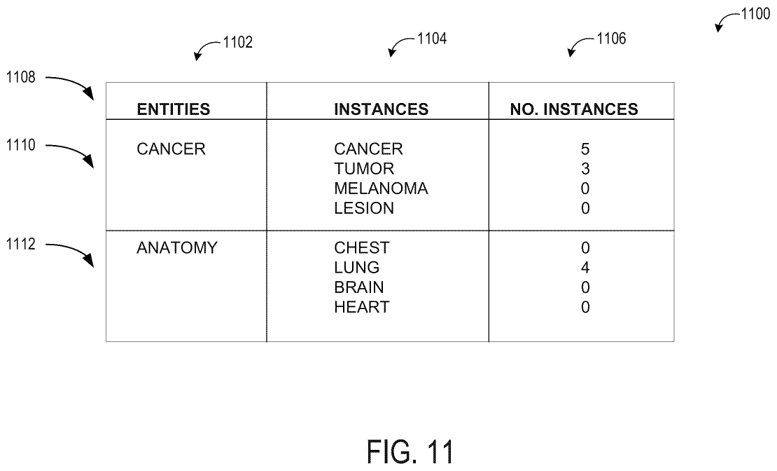

An example patient information system is shown in FIG. 1, which may include a plurality of entity recognition models used to generate a patient information summary. The plurality of entity recognition models may be trained on a respective plurality of labeled datasets based on a respective plurality of defined entities, as shown in FIG. 2A. The entity recognition models may be trained by following one or more steps of the method of FIG. 3. During an inference stage, the plurality of entity recognition models may label entities in a medical report, where outputs of the entity recognition models may be aggregated and refined to generate a patient summary, as described in reference to the diagram of FIG. 2B, in accordance with the high-level method shown in FIG. 4. The patient summary may include excerpts of labeled text taken from the medical report, as shown in FIGS. 8A and 8B. Labeling conflicts may occur where a text expression in the medical report is labeled differently by two or more different entity recognition models, which may be resolved by following one or more steps of the method of FIG. 5. Resolving the conflicts may include assigning relative weightings to outputs of the two or more different entity recognition models, as described in reference to the method shown in FIG. 6. To resolve a labeling conflict, outputs of the two or more different entity recognition models may be compared to an output of a multiple entity recognition model trained to label a plurality of entities in the medical report, by following one or more steps of the method shown in FIG. 7. Prior to aggregation, the multiple entity recognition model may output multiple candidate labels for a word or text expression, along with a probability vector including probability values indicating a relative probability of each candidate label being a correct identification of an instance of an entity, as shown in FIGS. 9A and 9B. An example excerpt of the patient summary after aggregation is shown in FIG. 10. In some embodiments, the patient summary may be generated more efficiently or faster by extracting entities/relations and storing them in one or more database tables where they may be quickly searched and retrieved, such as the database table shown in FIG. 11.

Embodiments of the present disclosure will now be described, by way of example, with reference to the figures, in which FIG. 1 schematically shows an example patient information system 100 that may be implemented in medical facility such as a hospital. Patient information system 100 may include a patient summary system 102. Summary system 102 may include resources (e.g., memory 130, processor(s) 132) that may be allocated to generate and store patient summaries for one or more medical reports drawn from one or more EMRs for each of a plurality of patients. For example, as shown in FIG. 1, summaries 106 and optionally medical reports 108 are stored on summary system 102 for a first patient (patient 1); a plurality of additional summaries and medical reports may be stored on and/or generated by summary system 102, each corresponding to a respective patient (patient 2 up to patient N).

Each summary 106 may include text and/or graphical representations of pertinent/relevant patient information associated with entities included in a given medical report. The entity-related information included in the summary 106 may include information related to disease, tissue, anatomy, problem, test, treatment, and/or other information included in the medical report and identified as being of interest.

The patient information that is presented via the summaries 106 may be stored in different medical databases or storage systems in communication with summary system 102. For example, as shown, the summary system 102 may be in communication with a picture archiving and communication system (PACS) 110, a radiology information system (RIS) 112, an EMR database 114, a pathology database 116, and a genome database 118. PACS 110 may store medical images and associated reports (e.g., clinician findings), such as ultrasound images, MRI images, etc. PACS 110 may store images and communicate according to the DICOM format. RIS 112 may store radiology images and associated reports, such as CT images, X-ray images, etc. EMR database 114 store electronic medical records for a plurality of patients. EMR database 114 may be a database stored in a mass storage device configured to communicate with secure channels (e.g., HTTPS and TLS), and store data in encrypted form. Further, the EMR database is configured to control access to patient electronic medical records such that only authorized healthcare providers may edit and access the electronic medical records. An EMR for a patient may include patient demographic information, family medical history, past medical history, lifestyle information, preexisting medical conditions, current medications, allergies, surgical history, past medical screenings and procedures, past hospitalizations and visits, etc. Pathology database 116 may store pathology images and related reports, which may include visible light or fluorescence images of tissue, such as immunohistochemistry (IHC) images. Genome database 118 may store patient genotypes (e.g., of tumors) and/or other tested biomarkers.

When requested, a summary 106 may be displayed on one or more display devices, such as a care provider device 134, and in some examples more than one care provider device, may be communicatively coupled to summary system 102. Each care provider device may include a processor, memory, communication module, user input device, display (e.g., screen or monitor), and/or other subsystems and may be in the form of a desktop computing device, a laptop computing device, a tablet, a smart phone, or other device. Each care provider device may be adapted to send and receive encrypted data and display medical information, including medical images in a suitable format such as digital imaging and communications in medicine (DICOM) or other standards. The care provider devices may be located locally at the medical facility (such as in the room of a patient or a clinician's office) and/or remotely from the medical facility (such as a care provider's mobile device).

When viewing summary 106 via a display of a care provider device, a care provider may enter an input (e.g., via the user input device, which may include a keyboard, mouse, microphone, touch screen, stylus, or other device) that may be processed by the care provider device and sent to the summary system 102. The user input may trigger display of the medical report that is summarized by the summary 106, trigger progression to a prior or future summary, trigger updates to the configuration of the summary, or other actions.

To generate the summaries 106, summary system 102 may include one or more entity recognition models 126. Each entity recognition model 126 may be a machine learning model, such as a neural network, trained to recognize one or more entities within a medical report of a patient, for example, received from an EMR. For example, a first entity recognition model may be trained to recognize each instance of a treatment mentioned in an EMR; a second entity recognition model may be trained recognize each instance of a disease mentioned in an EMR; a third entity recognition model may be trained to recognize each instance of a part of an anatomy of a subject; and so on.

To generate a summary, a medical report may be entered as input into each entity recognition model 126. Each entity recognition model 126 may then label instances of one or more entities in the medical report. In various embodiments, the entity recognition model 126 may also output, for each labeled entity, a probability that the entity is correctly and/or accurately labeled. For example, a first entity recognition model may be trained to recognize types of diseases. The first entity recognition model may label a first text expression “cancer” as the entity “disease”, with a first probability of 95% of the first text expression “cancer” being a disease. The first entity recognition model may label a second text expression “tumor” as the entity “disease”, with a second probability of 70% of the second text expression “tumor” being a disease. The first entity recognition model may label a third text expression “lesion” as the entity “disease”, with a third probability of 40% of the third text expression “lesion” being a disease, and so on. Separately, a second entity recognition model may be trained to recognize anatomical parts of a patient. The second entity recognition model may label a first text expression “lung” as the entity “anatomy”, with a first probability of 95% of the first text expression “lung” being a part of an anatomy of the patient. The second entity recognition model may label a second text expression “heart” as the entity “anatomy”, with a second probability of 95% of the second text expression “heart” being a part of an anatomy of the patient. The second entity recognition model may label a third text expression “aorta” as the entity “anatomy”, with a third probability of 70% of the third text expression “aorta” being a part of an anatomy of the patient, and so on.

The output from each entity recognition model may be aggregated and, in some examples, the aggregated output may be refined by applying one or more domain-specific rules, as will be explained in more detail below. The aggregated (and optionally refined) output may be saved and/or displayed as the summary.

Summary system 102 includes a communication module 128, memory 130, and processor(s) 132 to store and generate the summaries, as well as send and receive communications, graphical user interfaces, medical data, and other information.

Communication module 128 facilitates transmission of electronic data within and/or among one or more systems. Communication via communication module 128 can be implemented using one or more protocols. In some examples, communication via communication module 128 occurs according to one or more standards (e.g., Digital Imaging and Communications in Medicine (DICOM), Health Level Seven (HL7), ANSI X12N, etc.). Communication module 128 can be a wired interface (e.g., a data bus, a Universal Serial Bus (USB) connection, etc.) and/or a wireless interface (e.g., radio frequency, infrared, near field communication (NFC), etc.). For example, communication module 128 may communicate via wired local area network (LAN), wireless LAN, wide area network (WAN), etc. using any past, present, or future communication protocol (e.g., BLUETOOTH™, USB 2.0, USB 3.0, etc.).

Memory 130 one or more data storage structures, such as optical memory devices, magnetic memory devices, or solid-state memory devices, for storing programs and routines executed by processor(s) 132 to carry out various functionalities disclosed herein. Memory 130 may include any desired type of volatile and/or non-volatile memory such as, for example, static random access memory (SRAM), dynamic random access memory (DRAM), flash memory, read-only memory (ROM), etc. Processor(s) 132 may be any suitable processor, processing unit, or microprocessor, for example. Processor(s) 132 may be a multi-processor system, and, thus, may include one or more additional processors that are identical or similar to each other and that are communicatively coupled via an interconnection bus.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

One or more of the devices described herein may be implemented over a cloud or other computer network. For example, summary system 102 is shown in FIG. 1 as constituting a single entity, but it is to be understood that summary system 102 may be distributed across multiple devices, such as across multiple servers. Further, while the elements of FIG. 1 are shown as being housed at a single medical facility, it is to be appreciated that any of the components described herein (e.g., EMR database, RIS, PACS, etc.) may be located off-site or remote from the summary system 102. Further, the longitudinal data utilized by the summary system 102 for the summary generation and other tasks described below could come from systems within the medical facility or obtained through electronic means (e.g., over a network) from other referring institutions.

While not specifically shown in FIG. 1, additional devices described herein (e.g., care provider device 134) may likewise include user input devices, memory, processors, and communication modules/interfaces similar to communication module 128, memory 130, and processor(s) 132 described above, and thus the description of communication module 128, memory 130, and processor(s) 132 likewise applies to the other devices described herein. As an example, the care provider devices (e.g., care provider device 134) may store user interface templates in memory that include placeholders for relevant information stored on summary system 102 or sent via summary system 102. For example, care provider device 134 may store a user interface template for a patient timeline that a user of care provider device 134 may configure with placeholders for desired patient information. When the summary is displayed on the care provider device, the relevant patient information may be retrieved from summary system 102 and inserted in the placeholders. The user input devices may include keyboards, mice, touch screens, microphones, or other suitable devices.

FIG. 2A is a block diagram schematically illustrating an exemplary model training system 200 for a training a plurality of entity recognition models to each recognize respective entities in text data, such as in a medical report of a patient. For example, the medical report may be one of a plurality of medical reports or patient data files retrieved from an EMR of the patient (e.g., from EMR database 114 of FIG. 1). Entity recognition models 221 may be non-limiting examples of the entity recognition models 126 of FIG. 1. As described in greater detail with respect to FIGS. 3-9, a summary of the text data may be generated based on aggregated outputs of the plurality of entity recognition models 221. The summary may indicate which of the respective entities recognized by the plurality of entity recognition models is present in the text data, how often respective entities may be found, and other patient data associated with the respective entities.

Model training system 200 includes a plurality of defined entities 201, a plurality of labeled datasets 211, and a plurality of entity recognition models 221, where each of the respective pluralities are the same size. In other words, model training system 200 may include a number N defined entities 201, N labeled datasets 211, and N entity recognition models 221. Model training system 200 additionally includes a dataset curation block 210 and a model training block 220, which may represent modules or portions of code of a patient summary system (e.g., patient summary system 102) and/or processing stages including executing the portions of code and receiving input from human users of the patient summary system.

The plurality of entity recognition models 221 includes a first model 222, a second model 224, a third model 226, and so on, up to the total number N of entity recognition models 221. Similarly, the plurality of labeled datasets 211 includes a first dataset 212, a second dataset 214, a third dataset 216, and so on, up to the total number N of labeled datasets 211, and the plurality of defined entities 201 includes a first entity 202, a second entity 204, a third entity 206, and so on, up to the total number N of defined entities 201.

In various embodiments, each of the entity recognition models 221 may be trained at model training block 220 with a separate, differently labeled dataset 211, where each entity recognition model 221 is trained to identify a different defined entity 201 in the separate, differently labeled dataset 211. For example, dataset 212 may be created to train model 222 to identify or recognize instances of entity 202 in dataset 212; dataset 214 may be created to train model 224 to identify or recognize instances of entity 204 in dataset 214; dataset 216 may be created to train model 226 to identify or recognize instances of entity 206 in dataset 216, and so on.

In various embodiments, each of the entity recognition models 221 may be trained to additionally output, for each text expression labeled as an entity, a probability that the text expression is an instance of the entity (e.g., a confidence value). For example, model 222 may be trained to identify instances of “cancer” in dataset 212. A first text expression “tumor” may be labeled an instance of “cancer” with a probability of 95%. A second text expression “lesion” may be labeled an instance of “cancer” with a probability of 60%. As described in greater detail below, the probabilities may be used by a patient summary system such as patient summary system 102 to resolve labeling conflicts between different entity recognition models 221.

After training, during an inference stage, each of the entity recognition models 221 may receive new text data as input (e.g., extracted from a medical report of a patient), and may output labeled text data, where the labeled text data is the new text data with instances of one of the defined entities 201 labeled. In other words, model 222 may receive a medical report of a patient as input, and may output the medical report with labeled instances of entity 202 and associated probabilities; model 224 may receive the same medical report as input, and may output the medical report with labeled instances of entity 204 and associated probabilities; model 226 may receive the same medical report as input, and may output the medical report with labeled instances of entity 206 and associated probabilities; and so on. The inference stage is described in greater detail below in reference to FIG. 2B.

For example, the patient may be receiving treatment for cancer, and the medical report may refer to the cancer of the patient in various instances and in various ways. For example, the medical report may include the word “cancer” a number of times; it may also include words such as “tumor”, “melanoma”, “lesion”, and/or other similar words. Entity 202 may be “cancer”, where the words such as “tumor”, “melanoma”, and “lesion” are associated with and included within entity 202. Model 222 may be trained to identify entity 202 (e.g., instances of cancer and cancer-related expressions) in text data. To train model 222, dataset 212 may be created at dataset curation block 210, where dataset 212 includes various instances of the words such as “cancer”, “tumor”, “melanoma”, “lesion”, etc., that are labeled as “cancer”. Model 222 may be trained on dataset 212, and may not be trained on other datasets such as dataset 214 or dataset 216. Later, during an inference stage, the medical report may be entered as input into model 222, and model 222 may output a first version of the medical report where the words such as “cancer”, “tumor”, “melanoma”, “lesion”, etc., are labeled as the entity “cancer”.

In various embodiments, the words or text expressions may be labeled using a markup language. Specifically, to label a word, model 222 may insert a first markup tag immediately before the word, and may insert a second markup tag immediately after the word. For example, model 222 may receive the word “tumor” as input, and output text such as “<cancer>tumor</cancer>” to label tumor as being recognized as belonging to the entity “cancer”. In other embodiments, a different type of markup language, or a different type of identifier may be used to label words or text expressions associated with a defined entity. By labeling the entity with the markup language or different identifier, the word or text expression may be identified by the patient summary system. As described in greater detail below, labeling relevant text expressions as entities may allow the patient summary system to generate a patient summary that includes data related to, for example, a primary condition (e.g., cancer) of the patient. The patient summary system may perform various operations on the labeled words or text expressions to generate the patient summary. For example, the patient summary system may count a number of instances of an entity, and include the number of instances of the entity in the patient summary. The patient summary system may also include excerpts of one or more labeled medical reports, where the excerpts include instances of one or more entities. An example of an excerpt of a labeled medical report is shown in FIGS. 8A and 8B.

Similarly to model 222, model 224 may be trained to identify entity 204 (e.g., a different entity from entity 202) in text data, and model 226 may be trained to identify entity 206 (e.g., a different entity from entity 202 and entity 204). For example, entity 204 may be “anatomy”, and entity 206 may be “treatment”. To train model 224, dataset 214 may be created at dataset curation block 210, where dataset 214 includes various instances of the words such as “heart”, “lung”, “brain”, etc., that are labeled as “anatomy”. To train model 226, dataset 216 may be created at dataset curation block 210, where dataset 216 includes various instances of the words such as “chemotherapy”, “surgery”, etc., that are labeled as “treatment”. Model 224 may be trained on dataset 214, and may not be trained on other datasets such as dataset 212 or dataset 216. Model 226 may be trained on dataset 216, and may not be trained on other datasets such as dataset 212 or dataset 214.

In other embodiments, one or more of the entity recognition models 221 may be trained to identify and label instances of more than one entity 201. For example, model 222 may be trained to identify and label instances of entity 202 and entity 204, but not entity 206 or other entities; model 224 may be trained to identify and label instances of entity 204 and 206, but not entity 202; model 206 may be trained to identify and label instances of entity 202, entity 204, and entity 206; and so on. Some of the entity recognition models 221 may be trained to identify and label instances of a plurality of defined entities 201, and other entity recognition models 221 may be trained to identify and label instances of one entity.

In some embodiments, entity recognition models 221 that are trained to identify and label a plurality of entities 201 may be trained using labeled datasets 211 that are curated to train a model to identify and label a single entity. For example, if model 222 is trained to identify and label entity 202 and entity 204, model 222 may be trained using datasets 212 and 214, or datasets 212 and 214 may be aggregated or merged to form a new dataset that may be used to train model 222. In other embodiments where model 222 is trained to identify and label entity 202 and entity 204, model 222 may be trained using a new dataset different from and/or not including text data from datasets 212 and 214.

FIG. 2B shows a block diagram 250 schematically illustrating a flow of data when generating a patient information summary from a medical report using the trained entity recognition models 221 of FIG. 2A. Block diagram 250 includes a medical report 252, which may be processed by a patient summary system 254 (e.g., patient summary system 102) to generate a patient summary 262. Patient summary system 254 may include a model output aggregation block 256, an output refinement block 258, and a summary generation block 260, which may represent modules or portions of code of patient summary system 254 and/or processing stages including executing the portions of code and receiving input from human users of patient summary system 254.

Patient summary system 254 may enter medical report 252 as input into one or more entity recognition models 221 (e.g., model 222, model 224, model 226, etc.). Each of the one or more entity recognition models 221 may output a version of medical report 252, with labeled instances of an entity on which the corresponding entity recognition model was trained. Outputs of the one or more entity recognition models 221 may then be aggregated at model output aggregation block 256. The aggregation of the outputs may be performed by following one or more steps of a procedure described in relation to FIG. 5. An output of model output aggregation block 256 may be a labeled version of medical report 252, where instances of a plurality of entities are labeled. Each instance of the plurality of entities may correspond to a respective entity of each of the one or more entity recognition models 221. For example, the one or more entity recognition models 221 may include models 222, 224, and 226, and the labeled version of medical report 252 may include text expressions labeled as entity 202, entity 204, and entity 206.

Aggregating the outputs of the entity recognition models 221 may include resolving any labeling conflicts between different entity recognition models. In some cases, a word in medical report 252 may be labeled differently by more than one model. For example, model 222 may be trained to label instances of diseases, and model 224 may be trained to label instances of anatomical parts of a patient, as in the example described above. Medical report 252 may include the expression “lung cancer”. The word “lung” in expression “lung cancer” may be labeled as a disease by model 222, and may be labeled as anatomy by model 224. During aggregation of the output of model 222 and model 224, the word “lung” in expression “lung cancer” may be resolved as being either a disease or an anatomical part of the patient. In other words, a single entity label may be associated with multiple labels, where each entity label is the model output that has the highest probability of being accurate. Resolving conflicts between entity labels may involve determining a relative weighting of different model outputs, as described below in reference to FIG. 6. In other embodiments, a plurality of entity labels may be associated with a single word or text expression.

After the outputs of the entity recognition models 221 have been aggregated, the labeled version of medical report 252 where instances of a plurality of entities are labeled may be further refined by output refinement block 258. Refining the aggregated, labeled version of medical report 252 may include adjusting or changing one or more entity labels based on context-based clinical knowledge and/or natural language processing (NLP), as described in greater detail below in reference to FIG. 7.

After refining the aggregated, labeled version of medical report 252, a patient summary 260 may be generated by summary generation block 260 of patient summary system 254. Generation of the patient summary 260 is described below in reference to FIG. 4.

Referring now to FIG. 3, an exemplary method 300 is shown for training a plurality of entity recognition models to recognize predefined entities in text data, as part of a patient summary system. The entity recognition models described in method 300 and other methods included herein may be non-limiting examples of the entity recognition models 126 of FIG. 1 and the entity recognition models 221 of FIGS. 2A and 2B. As such, method 300 and the other methods included herein may be described in reference to patient summary system 102, model training system 200, and/or block diagram 250 of FIGS. 1, 2A, and 2B, respectively. In various embodiments, method 300 and the other methods included herein may be carried out by processor(s) 132 of patient summary system 102.

Method 300 begins at 302, where method 300 includes selecting a set of desired entities around which various patient summaries may be generated (e.g., defined entities 201). The desired entities may be a set of classes or types into which words or text expressions commonly found in medical records of a patient may be classified, which may represent areas of interest to a caregiver or healthcare professional reviewing an EMR of the patient. For example, the desired entities may include concepts such as disease, anatomy, problem, test, tissue, treatment, diagnosis, and the like. In some embodiments, the desired entities may be structured in a hierarchical manner, where an entity of the desired entities may include one or more categories. For example, the entity “cancer” may include categories for different types of cancer, such as lung cancer, skin cancer, colon cancer, etc. Further, the categories may additionally include one or more levels of subcategories. In some embodiments, the set of desired entities may be large and/or comprehensive, where the patient summary system may generate patient summaries that may summarize a wide range of patient data. In other embodiments, the set of desired entities may be smaller, and the patient summary system may generate patient summaries that are smaller and/or focused on specific types of patient data or conditions. For example, in one embodiment, the patient summary system may generate patient summaries with respect to a single medical condition, such as diabetes, and may display summarized data of the patient associated with diabetes, such as blood sugar levels, cholesterol levels, etc.

At 304, method 300 includes, for each entity of the set of desired entities, creating a pair of datasets including an input dataset and a labeled dataset to be used as ground truth data to train an entity recognition model to recognize and label the corresponding entity. In other words, the entity recognition model may be trained on two datasets: an input dataset without any entity labels, and a corresponding ground truth dataset including the same text data as the input dataset, where instances of the corresponding entity have been labeled.

In various embodiments, each pair of datasets may be different. For example, each labeled dataset of each pair may be created using a different process. Each labeled dataset may be curated in a different manner, to achieve different desired target characteristics, by different human experts. Each pair of datasets may be stored in a different location. For example, a first pair of datasets may be stored in a first database; a second pair of datasets may be stored in a second database; and a third pair of datasets may be stored in a third database, where each of the first, second, and third databases may be internal or external to the patient summary system or stored in a different location in a memory of the patient summary system (e.g., memory 130).

At 306, creating the pair of datasets includes selecting relevant text data from various sources. The various sources may include, for example, anonymized historical patient reports or records extracted from EMRs of a set of patients, where the reports or records include a variety of instances of the entity. For example, the entity may be “cancer”, and the records may be selected from patients who suffer from cancer, where the records include a plurality of different terms describing cancer. The records may be selected from patients suffering from cancer in addition to one or more different medical conditions, to train the corresponding entity recognition model to recognize the plurality of different terms describing cancer as cancer, and not recognize the plurality of different terms describing cancer as the different medical condition, and not recognize various terms describing the different medical condition as cancer. In other embodiments, the various sources may include, without limitation, publicly available datasets, anonymized medical reports from the hospitals, synthetically generated datasets, and so on.

At 308, creating the pair of datasets includes combining and curating the selected relevant text data to achieve a target frequency of instances of the entity, where the instances have a target length and achieve a target adjacency. In various embodiments, curation of the selected relevant text data may be performed at least partially by human experts, including doctors and/or engineers skilled in the art of building models. The desired target characteristics may be selected to increase or maximize an efficiency of training a corresponding entity recognition model. The desired target characteristics may include a frequency of instances of the entity. For example, for the entity “cancer”, the text data may be curated such that a number of instances of the word “cancer” are included that is within a first target range of numbers. The text data may be curated such that a number of instances of words like “tumor”, “lesion”, etc. are included that is within other target ranges of numbers, which may be the same as the first target range or different from the first target range. The text data may be curated for an adjacency of the instances of words or text expressions referring to cancer. For example, the text data may be edited such that the instances of words or text expressions referring to cancer are distributed in a balanced and even manner throughout the text data, and are not distributed in such a way that the instances are concentrated in portions of the text data. A length of the instances (e.g., a length of text expressions longer than a single word) may be analyzed, for example, to ensure that instances have lengths that facilitate training the entity recognition model efficiently. It should be appreciated that the examples provided herein are for illustrative purposes, and a greater or lesser number of different types of curation may be used to curate the text data without departing from the scope of this disclosure.

The desired target characteristics may be different for different labeled data sets and/or different entities. Relevant text data may be abundant for some entities, and more scarce for other entities. For example, text data for an entity “anatomy” may be easily found in a large number of medical reports; text data for an entity “cancer” may be found in a smaller number of medical reports; text data for an entity “tissue” may be found in an even smaller number of medical reports; and so on. As a result, the labeled datasets may be of different sizes. As a result of the datasets being different sizes, the corresponding entity recognition models may not train or perform equally well. For example, a first model trained on a first pair of large datasets may achieve a first performance on a first medical report, and a second model trained on a second pair of smaller datasets may achieve a second performance on a second medical report, where the second performance is lower than the first performance. The first medical report may be the same as the second medical report, or the first medical report may be different from the second medical report.

At 310, creating the pair of datasets includes labeling the combined and curated text data to generate the labeled dataset. Labeling the combined and curated text data may include various manual and/or automated steps. For example, one or more human experts may compile a list of instances of words or text expressions to be labeled that are found in the curated text data. A computer program may be written to insert markup language into the curated text data to label each instance of the list of instances. In some embodiments, a computer application of the patient summary system may be configured to take the list of instances as input, and automatically generate the labels.

It should be appreciated that while steps 306, 308, and 310 describe creating individual labeled datasets each for a single entity, one or more labeled datasets may be created for training a multiple entity recognition model by following a similar procedure. The similar procedure may include selecting, from the various sources, relevant text data including instances of a plurality of entities; combining and curating the text data as described above, with target frequencies, lengths, and adjacency of the plurality of entities; and labeling the instances of the text data to form a labeled dataset including labeled instances of more than one entity.

At 312, method 300 includes training an entity recognition model on each pair of datasets. In various embodiments, the entity recognition model may be provided with the input dataset and ground truth dataset (e.g., the labeled dataset) as input. The entity recognition model may output a labeled version of the input dataset, based on a set of parameters of the entity recognition model. The set of parameters may be adjusted by applying a gradient descent algorithm, and back-propagating a difference (e.g., error) between an output of the entity recognition model and the ground truth dataset through the network to minimize the difference.

As described above, the output may be a labeled version of the input dataset where each label includes a probability value of the model accurately identifying an instance of an entity. In the case of a multiple entity recognition model, the output may be a labeled version of the input dataset where each label includes a probability vector comprising a plurality of probability values (e.g., one for each entity on which the multiple entity recognition model was trained).

When the multiple entity recognition model identifies an instance of an entity of a number of entities on which the model has been trained, the multiple entity recognition model may output a probability vector for the instance, where the probability vector includes a probability of each entity being the most accurate entity to assign to the instance. Put another way, each probability may be a confidence level of the multiple entity recognition model in each possible entity into which the instance may be classified. The probability vector may include a number of probability values that corresponds to the number of entities on which the model has been trained.

For example, if the multiple entity recognition model is trained to recognize instances of five entities in text data, the multiple entity recognition model may output, for each word or expression recognized as being an instance of at least one of the five entities, a probability vector comprising five probability values. A first probability value of the probability vector may indicate a probability of the word or expression being an instance of a first entity of the five entities; a second probability value of the probability vector may indicate a probability of the word or expression being an instance of a second entity of the five entities; a third probability value of the probability vector may indicate a probability of the word or expression being an instance of a third entity of the five entities; a fourth probability value of the probability vector may indicate a probability of the word or expression being an instance of a fourth entity of the five entities; and a fifth probability value of the probability vector may indicate a probability of the word or expression being an instance of a fifth entity of the five entities.

Additionally, during a later inference stage, a patient summary system may determine a highest probability value of the five probability values, and assign a label to the word or expression classifying the word or expression as an instance of the entity with the highest probability value. For example, the multiple entity recognition model may be trained to identify two different entities, an entity “disease” and an entity “anatomy”. An expression “lung cancer” in the medical report may be identified by the multiple entity recognition model as an instance of either or both of the entity “disease” and the entity “anatomy”. The entity recognition model may output a probability vector including a first probability of “lung cancer” being accurately labeled as a “disease” of 80%, and a second probability of “lung cancer” being accurately labeled as “anatomy” of 20%. As a result of the first probability (80%) being greater than the second probability (20%), “lung cancer” in the medical report may be labeled as an instance of the entity “disease”, and may not be labeled as an instance of the entity “anatomy”. An example of a probability vector associated with an entity is described in reference to FIG. 9A below.

It should be appreciated that in some examples, training may occur via a system external to the patient summary system, and the trained models may then be stored in the patient summary system.

At 314, method 300 includes storing the trained models for deployment (e.g., in memory 130), and method 300 ends.

Referring now to FIG. 4, an exemplary method 400 is shown for generating a patient information summary of a medical report of a patient, using a plurality of trained entity recognition models, within a patient summary system such as patient summary system 102.

Method 400 begins at 402, where method 400 includes receiving a medical report. In various examples, the medical report may be retrieved from an EMR of the patient (e.g., EMR database 114). For example, a caregiver such as a doctor of the patient may retrieve the medical report from the EMR, and input the medical report into the patient summary system, which may output the patient information summary on a display device of the patient summary system (e.g., care provider device 134).

At 404, method 400 includes selecting one or more desired entities to be labeled by the plurality of trained entity recognition models. The one or more desired entities may be related to a condition of the patient of interest to the caregiver. For example, the patient may be suffering from cancer, and the caregiver may wish to review information of the medical report related to cancer, such as diagnoses, treatments, historical data, etc. The patient may additionally be suffering from other conditions. If the other conditions are of interest to the caregiver, entities related to the other conditions may be included in the selected one or more desired entities. For example, if the patient is suffering from diabetes and cancer, and the caregiver is interested in patient information regarding both the diabetes and the cancer, the one or more desired entities to be labeled by the trained entity recognition models may include a first entity cancer, and a second entity diabetes. If the caregiver is not interested in the other conditions of the patient, the one or more desired entities may include the first entity cancer, and may not include the second entity diabetes and/or other entities related to the other conditions.

At 406, method 400 includes inputting the medical report into one or more entity recognition models corresponding to the one or more desired entities. For example, the medical report may be inputted into a first entity recognition model corresponding to the entity cancer. The first entity recognition model may output a first version of the medical port in which instances of cancer expressions are labeled with the entity cancer. The medical report may also be inputted into a second entity recognition model corresponding to the entity diabetes, and the second entity recognition model may output a second version of the medical report in which instances of diabetes expressions are labeled with the entity diabetes. In this way, a plurality of entity recognition models may be employed to label various entities in the medical report, where each entity recognition model of the plurality of entity recognition models outputs a differently labeled version of the medical report.

Additionally or alternatively, as described above, the medical report may be entered as input into one or more multiple entity recognition models corresponding to a plurality of the one or more desired entities. For example, the medical report may be entered into a first multiple entity recognition model trained to label instances of bold cancer expressions and diabetes expressions (e.g., trained on a labeled data set including labeled instances of both cancer expressions and diabetes expressions). The first multiple entity recognition model may output a third version of the medical report in which instances of both cancer expressions and diabetes expressions are labeled with the entities cancer and diabetes, respectively. If additional multiple entity recognition models are available for additional entities, the medical report may be entered into the additional multiple entity recognition models.

At 408, method 400 includes aggregating the labeled model output and resolving any entity conflicts. Aggregating the labeled model output may include merging a plurality of versions of the medical report, where in each version instances of one or more entities are labeled as such. When the plurality of versions are merged, one or more labeled words or text expressions may be labeled differently by different entity recognition models. In some scenarios, the entities used to train the entity recognition models may be may be mutually exclusive, where multiple labels may not be used for a single entity. In other scenarios, the entities used to train the entity recognition models may not be mutually exclusive, and multiple labels may be used for a single entity. For example, a first entity recognition model may be trained to label instances of the entity “procedure”, and a second entity recognition model may be trained to label instances of the entity “treatment”, where an instance of the word “surgery” may be labeled as a procedure by the first entity recognition model and as a treatment by the second entity recognition model. As another example, an entity “cancer” may include a subcategory “lung cancer”. An expression “tumor in lung” may be labeled both as “cancer” and “lung cancer”, or may be labeled as “cancer” and “anatomy”.

A labeling conflict may occur when a text expression is labeled as two or more mutually exclusive entities. When a labeling conflict occurs, the conflict may be resolved by selecting a most accurate entity label. Resolving labeling conflicts is described in greater detail below, in reference to FIG. 5.

At 410, method 400 includes refining the aggregated model output (e.g., after conflicts have been resolved). Refining the aggregated model output may include using additional internal or external resources to determine whether one or more entities are properly labeled. For example, a word in a labeled, merged version of the medical report may be labeled as a first entity, when the word would be more appropriately labeled as a second, different entity. If the word would be more appropriately labeled as the second, different entity, the label of the word may be changed from the first entity to the second entity.

At 412, refining the aggregated model output includes adjusting or changing one or more labels of the aggregated labeled model output based on clinical context-based knowledge from one or more domain specific tools. The one or more domain specific tools may include, for example, a unified medical language system, medical subject headers, one or more medical dictionaries, databases, ontologies, or other similar resources. The one or more domain specific tools may include public or private online resources, and/or resources that are internal to the patient summary system or available to the patient summary system via one or more hospital or healthcare networks that the patient summary system is connected to. In various embodiments, words or multi-word expressions that are labeled in the aggregated labeled model output may be consulted in the domain specific tools to determine whether a more accurate label may exist. If a more accurate label exists, the label may be changed.

As one example, the word “melanoma” may be labeled as a first entity “cancer” in the aggregated labeled model output. “Melanoma” may be looked up in an online medical dictionary. An identifier (e.g., an alphanumeric code) of the term “melanoma” in the online medical dictionary may be extracted. A search for the identifier may be performed on one or more additional online resources, which may return a set of possible synonyms for melanoma. The set of possible synonyms may be reviewed to determine if one or more of the synonyms may also be an entity defined by the patient summary system (e.g., the defined entities 201). One of the synonyms may be a second entity “skin cancer”, which may be a subcategory of the entity “cancer”. The second entity “skin cancer” may be compared with the first entity “cancer” to determine a most accurate classification for the word “melanoma”. The second entity “skin cancer” may be determined to be more accurate classification of “melanoma”. For example, in one embodiment, “skin cancer” may automatically be determined to be a more accurate classification as a result of “skin cancer” being a subcategory of the entity “cancer” (e.g., where a more specific term is considered a more accurate classification than a less specific term). In other embodiments, a different procedure may be used to assess an accuracy of an entity label. As a result of being determined to be more accurate, the first entity label “cancer” may be replaced with the second entity label “skin cancer” in the aggregated model output (e.g., the labeled version of the medical report.

As another example, the expression “lung cancer” may be labeled as the entity “disease” by an entity recognition model and as “cancer diagnosis” by another entity recognition model. An entry for “cancer diagnosis” in the medical dictionary may include a parent concept called “disease. As a result of identifying one entity type as being a parent concept compared to the other, the entity “cancer diagnosis” may be determined to be a more granular classification for “lung cancer” than “disease”, whereby the label “disease” may be replaced by the label “cancer diagnosis”.

At 414, in some embodiments, refining the aggregated model output may include adjusting or changing one or more labels of the aggregated labeled model output based on grammar-based rules. Natural language processing (NLP) may be performed on sentences of the aggregated model output, where words adjacent to, near, or surrounding a labeled entity may be analyzed to determine if the entity is accurately labeled. For example, an adjective of a labeled word may indicate that an entity label is incorrect.

At 416, method 400 includes generating a summary of the labeled version of the medical report from the aggregated labeled text data outputted by the model, where the summary summarizes patient information related to the one or more desired entities. To generate the summary, the patient summary system may extract instances of the desired entities, which may be identified by labels as described above, and generate text content based on the entities to display to a caregiver. The text content may include, for example, numbers and types of entities and instances included in the medical report, excerpts of labeled text of the medical report, and/or additional patient data relating to the extracted entities.

In various embodiments, in addition to generating the text content, the extracted instances of the desired entities may be assembled into a data structure, where the data structure may be faster and more efficient for the patient summary system to search than the labeled text content. The extracted instances may be assembled into the data structure prior to generating the labeled text content, and the data structure may be used to generate the text content, or the extracted instances may be assembled into the data structure during or after generating the labeled text content. For example, the caregiver may enter a set of desired entities into the patient summary system, and the patient summary system may enter each of the desired entities into a respective entity recognition model. Outputs of the respective entity recognition models may be aggregated and refined as described above, to generate the labeled text content. The instances of the desired entities in the labeled text content may be assembled into the data structure. The patient summary system may select, for example, via a stored configuration or preference of the user, a desired format of the patient summary. The patient summary system may search for the instances of the desired entities in the data structure, and may generate the patient summary in accordance with the desired format, based at least partially on data retrieved from the data structure. Because the data structure may be searched more quickly and efficiently than the labeled text content, a speed with which the patient summary may be generated may be increased. For example, the desired format may include a list of instances of a primary entity of the desired entities, and the instances of the primary entity may be searched for and retrieved from the data structure more quickly than searching for and retrieving the instances from the labeled text content.

In some embodiments, the data structure may be a hierarchical data structure, where the extracted instances of the desired entities may be organized in a hierarchical manner. In other embodiments, the data structure may be configured in a different manner, for example, to facilitate efficient searching in accordance with one or more search algorithms known in the art. In various examples, the data structure may be a relational database.

Referring briefly to FIG. 11, an exemplary database table 1100 of a relational database (e.g., a data structure as described above) is shown, where database table 1100 includes three columns and three rows. A first column 1102 of database table 1100 includes a plurality of desired entities selected to be labeled in the text content; a second column 1104 includes a plurality of instances of each of the desired entities of first column 1102; and a third column 1106 includes a number of each of the instances included in second column 1104. A first row 1108 of database table 1100 includes column headings for columns 1102, 1104, and 1106; a second row 1110 of database table 1100 includes data for the entity “cancer”; and a third row 1112 of database table 1100 includes data for the entity “anatomy”. Using database table 1100, the patient summary system may more quickly and efficiently retrieve information about entities in the text content than by automated parsing of the text content. For example, a user of the patient summary system may wish to see a listing of all instances of the entity “cancer” in the text content. The patient summary system may request a list of instances of the entity “cancer” found in the text content from the relational database for which a number of instances is greater than 0. The entity “cancer” in database table 1100 may be consulted, and based on the information included in row 1110, data may be retrieved indicating that five instances of the word “cancer” were found in the text content, and three instances of the word “tumor”. The patient summary system may display the data in a patient summary directed at the user.

Returning to method 400, a format and a content of the summary may vary over different embodiments. The format and content of the summary may be configured, for example, by one or more care providers, or by one or more administrators of a hospital, or by a different medical professional. In some embodiments, the format and/or content may be customizable to a specific care provider. For example, a first care provider may wish to see a first set of patient data in a first summary, and a second care provider may wish to see a second, different set of patient data in a second, different summary. Alternatively, the first care provider and the second care provider may wish to see the same patient data, but the first care provider may wish to format the patient data in a first way, and the second care provider may wish to format the patient data in a second way. For example, the first care provider may prefer a first set of entities highlighted, and the second care provider may prefer a second set of entities highlighted.

The summary may include a listing of one or more entities labeled in the labeled medical report. For example, a care provider may select, via a user interface (UI) of the patient summary system (e.g., a UI of care provider device 134), to view a summary of the patient data based on the entities “cancer” and “treatment”. The summary generated may include a listing of the selected entities “cancer” and “treatment”. In some embodiments, the summary may include a count of a number of entities recognized and labeled in the labeled medical report. For example, the medical report may include 10 labeled instances of the entity “cancer” (e.g., cancer, tumor, lesion, etc.) and four instances of the entity “treatment”. The summary may include a statement indicating that the 10 labeled instances of cancer and the four instances of treatment were detected. The summary may include a listing of the words or expressions labeled as “cancer” and “treatment” identified in the labeled medical report. The content of the summary may be organized and displayed as bulleted lists, or the content of the summary may be expressed in sentences or paragraphs that may be pre-configured.

In various embodiments, the summary may include excerpts from the labeled medical report. The excerpts may include individual sentences, portions of sentences, groups of sentences, or whole paragraphs of the labeled medical report. In one embodiment, an excerpt may include all the text in the labeled medical report. The excerpts may be displayed on the display device with some or all of the labels indicated. For example, a name of an entity may be displayed next to a labeled instance of the entity. The name of the entity and/or the labeled instance may be highlighted. For example, either or both of the entity name and the labeled instance may be included in bold text, or in italics, or in a different format. Either or both of the entity name and the labeled instance may be highlighted, for example, in the same or different colors. For example, a first entity name may be highlighted in a first color, and a second entity name may be highlighted in a second color.

At 418, method 400 includes displaying the summary and/or the aggregated labeled text data outputted by the model on a display device of the patient summary system (e.g., care provider device 134). At 420, method 400 includes storing the summary and/or the aggregated labeled text data outputted by the model in the patient summary system (e.g., in summaries 106). In various embodiments, either or both of the summary and the aggregated labeled text data may be used by various downstream applications. Method 400 ends.

Referring briefly to FIG. 8A, a model output example 800 shows an exemplary excerpt 802 of an output of an entity recognition model. The entity recognition model may be a non-limiting version of model 222 of FIGS. 2A and 2B, where the entity recognition model may be trained to identify instances of the entity “cancer”. For example, the entity recognition model may be trained on dataset 212 of FIG. 2A.

In the depicted embodiment, excerpt 802 includes the word “tumor”, which has been labeled as an instance of the entity “cancer” by the entity recognition model. Specifically, the entity recognition model has inserted the markup tags <cancer> and </cancer> to identify the word “tumor” as cancer. A probability of the word “tumor” being accurately identified as “cancer” is also included, as described above. When generating the patient summary, a module of the patient summary system may search for the markup tags. When the markup tags are encountered, executable code of the module may replace the tagged entity with a graphical label, as shown in FIG. 8B.

In FIG. 8B, a summary display example 850 shows an exemplary display excerpt 852 generated from exemplary excerpt 802 of model output example 800 of FIG. 8A, where display excerpt 852 is displayed within a patient summary generated by a patient summary system (e.g., patient summary system 102), in accordance with an embodiment. Executable code of the patient summary system may detect markup tags in excerpt 802 of FIG. 8A, and insert a graphical label at a location of the markup tags. The graphical label may include formatting and/or highlighting such as, for example, a colored/shaded background, bold text, colored text, or other visual features to indicate the relevant entity. In some embodiments, the formatting and/or highlighting may be customized based on the probability value assigned by the entity recognition model.

Additionally, the formatting and/or highlighting may be specific to the entity. For example, a first entity recognition model may include a first formatting and/or highlighting for identifying a first entity; a second entity recognition model may include a second formatting and/or highlighting for identifying a second entity; and so on. In this way, when outputs of a plurality of entity recognition models are aggregated, each entity recognized by a respective entity recognition model may be indicated in a distinctive manner. An example of an excerpt of a patient summary with aggregated model outputs is described below in reference to FIG. 10.

Similar to FIG. 8A, FIG. 9A includes a model output example 900 showing a first exemplary excerpt 902 and a second exemplary excerpt 904 of an output of a multiple entity recognition model, where the model output includes a probability vector including probability values for each entity on which the multiple entity recognition model is trained. The multiple entity recognition model is trained on two entities: “cancer”, and “anatomy”. For example, the multiple entity recognition model may be trained on a dataset including labeled cancer entities, and labeled anatomical parts.

As in FIG. 8A, excerpt 902 includes the word “tumor”, which has been labeled as an instance of the entity “cancer” by the entity recognition model. Specifically, the entity recognition model has inserted the markup tags <cancer> and </cancer> to identify the word “tumor” as cancer. A probability vector of the word “tumor” is also included, where the probability vector includes three probability values relating to the entities “cancer” and “anatomy”, and a probability of “tumor” not being identified as “cancer” or “anatomy”, in that order. A first probability value of 80% indicates a probability of “tumor” being identified as “cancer”. A second probability value of 10% indicates a probability of “tumor” being identified as “anatomy”. A third probability of 10% indicates a probability of “tumor” being identified as “outside” (e.g., a non-cancer and non-anatomy entity). As a result of the probability of “tumor” being identified as “cancer” being greater than the probability of “tumor” being identified as “anatomy” (or “outside”), the markup tags <cancer> and </cancer> are selected to label “tumor” as “cancer”.

Similarly, the expression “frontal lobe” has been labeled an instance of the entity “anatomy”, as a result of being assigned a greater probability than “cancer” and “outside” (e.g., 80% vs. 10% vs 10%).

In second excerpt 904, in accordance with the model output, the expression “brain tumor” has a 60% probability of being an instance of “cancer”, a 30% probability of being an instance of “anatomy”, and a 10% chance of being “outside”, as a result of the inclusion of the word “brain”. As a result of “brain tumor” having a higher probability of being an instance of “cancer” than being an instance of “anatomy”, the multiple entity recognition model includes the markup tags for “cancer”, while the probability vector includes information that “anatomy” is also a possibility.

In FIG. 9B, a summary display example 950 shows a first exemplary display excerpt 952 and a second exemplary display excerpt 954 generated from exemplary excerpts 902 and 904 of model output example 900 of FIG. 9A, where display excerpts 952 and 954 are displayed within a patient summary generated by a patient summary system (e.g., patient summary system 102), in accordance with an embodiment.

Exemplary display excerpt 952 may be displayed on a screen to a caregiver (e.g., on care provider device 134). When generating the patient summary, a module of the patient summary system may search for the markup tags for “cancer” and “anatomy”. When the markup tags are encountered, executable code of the module may replace a respective tagged entity with a respective graphical label. As described in reference to FIG. 8B, the graphical label may include formatting and/or highlighting such as, for example, a colored/shaded background, bold text, colored text, or other visual features to indicate the relevant entity. The formatting and/or highlighting may be customized based on the probability value assigned by the entity recognition model.

In second excerpt 954, labels for “cancer” and “anatomy” may be both included for the word “brain tumor”, due to a difference between the probabilities (e.g., 60%, 30%, 10%, from FIG. 9A) being below a threshold difference. Additionally, the labels for “cancer” and “anatomy” may be visually distinguished from each other based on the difference in probabilities. For example, the label “cancer” may be displayed with a first formatting (e.g., in white), and the label “anatomy” may be displayed with a second formatting (e.g., in a darker shade). In this way, an uncertainty of the model output may be communicated to the caregiver. It should be appreciated that in other embodiments, different types of labeling techniques and/or different types of formatting and/or highlighting may be used.

FIG. 10 shows a third exemplary excerpt 1000 of a labeled medical report generated based on outputs of a plurality of entity recognition models, where the third exemplary excerpt is displayed on a display of a patient summary system such as patient summary system 102 of FIG. 1. In excerpt 1000, various text expressions are labeled as instances of entities identified by the plurality of entity recognition models. The label of a text expression may be different depending on a labeled entity of the text expression. For example, instances of a first entity may be labeled in a first color, shading, or formatting; instances of a second entity may be labeled in a second color, shading, or formatting; and so on. In this way, a caregiver viewing third exemplary excerpt 1000 (e.g., in a patient summary) may quickly scan for one or more desired entities.

For example, a first entity “cancer” may be displayed in the first color, such that a caregiver interested in the first entity “cancer” may quickly scan excerpt 1000 for labels of the first color. A second entity “gene or geneproduct” may be displayed in a second color, which may be different from the first color. A third entity “multi-tissue structure” may be displayed in a third color, which may be different from the first and second colors.

Returning to FIG. 4, the summary may also include additional data of the patient. For example, the patient summary may include, for each treatment entity detected, treatment information included in the medical report. Sentences describing the treatments may be identified based on specific content of the sentences, and the sentences may be included in the patient summary. For example, the patient summary system may scan for the labeled medical report for sentences near a labeled instance of a treatment that include starting and/or ending dates and/or times of the treatment, which may be extracted and displayed in the summary.

In some embodiments, the additional data may not be included in the medical report, and may be extracted from a different source, such as an EMR of the patient. For example, the patient summary system may determine a name and/or identifier of the patient in the medical report. The patient summary system may conduct a search for the name and/or identifier in an EMR database (e.g., EMR database 114). The patient summary system may access the EMR of the patient, and retrieve patient data from the EMR. The patient data may include, for example, admission data, historical patient data, administrative data such as location data of the patient, and/or any other information of the patient. The patient data may be displayed in the summary along with the entity information. It should be appreciated that the examples provided herein are for illustrative purposes, and various different types and/or amounts of information may be included in the patient summary, in a variety of different formats, without departing from the scope of this disclosure.

Referring now to FIG. 5, an exemplary method 500 is shown for aggregating labeled model output of a plurality of trained entity recognition models, where aggregating the labeled model output includes resolving entity conflicts. The entity recognition models may be non-limiting examples of the entity recognition models 221 of FIGS. 2A and 2B, within a patient summary system such as patient summary system 102 of FIG. 1. The labeled model output of the plurality of trained entity recognition models may be generated as a result of inputting a medical report of a patient into the plurality of trained entity recognition models.

Method 500 begins at 502, where method 500 includes receiving a labeled medical report including labeled entities from each entity recognition model of the plurality of trained entity recognition models. In various embodiments, the labeled medical report may be generated by following the procedure described in reference to FIG. 4.

At 504, method 500 includes proceeding through the labeled medical report and reviewing each labeled instance of an entity one by one, to determine whether more than one entity label has been assigned to the labeled instance. For example, a first entity label may be assigned to the labeled instance by a first entity recognition model of the plurality of entity recognition models, and a second entity label may be assigned to the labeled instance by a second entity recognition model of the plurality of entity recognition models.

At 506, method 500 includes determining whether the instance of the entity was labeled as more than one different entity by two or more of the entity recognition models. The instance may be labeled as more than one different entity when different labels are assigned to the instance by the two or more entity recognition models, and the different labels are mutually exclusive (e.g., for example, not a category and an appropriate subcategory). If at 506 it is determined that the instance was labeled as more than one different entity by two or more entity recognition models, method 500 proceeds to 508.

At 508, method 500 includes assigning a relative weighting to outputs of the two or more entity recognition models, and selecting a most accurate entity label of the different labels based on the relative weightings. Selecting the most accurate entity label of the different labels is described in greater detail below in reference to FIG. 6.

Alternatively, if at 506 it is determined that the instance was not labeled as more than one different entity by two or more entity recognition models, method 500 proceeds to 510. At 510, method 500 includes accepting the label assigned by the multiple entity recognition model, and method 500 ends.

Referring now to FIG. 6, an exemplary method 600 is shown for assigning a label to an instance of an entity in a medical report based on a relative weighting of an output of a plurality of entity recognition models receiving the medical report as input, within a patient summary system such as patient summary system 102.

Method 600 begins at 602, where method 600 includes assigning initial weightings to outputs of the plurality of entity recognition models based on probability values or vectors outputted by each entity recognition model. For entity recognition models trained on a single entity, the probability value is a probability of the output of the entity recognition model correctly identifying the instance. For a multiple entity recognition model trained on a plurality of entities, the probability vector includes relative probabilities of a labeled expression being an instance of each of the plurality of entities.

As a first example, the medical report may include the expression “lung cancer”. A first entity recognition model trained to identify instances of the entity “cancer” may label the expression “lung cancer” as “cancer”, with a first probability. A second entity recognition model trained to identify instances of the entity “anatomy” may label the expression “lung cancer” as “anatomy”, with a second probability. To resolve the conflict between the output of the first entity recognition model and the second entity recognition model with respect to labeling “lung cancer”, relative weightings of the two model outputs may be assigned based on the probabilities. If the first probability is higher than the second probability, the output of the first entity recognition model may be weighted higher than the output of the second entity recognition model. If the first probability is lower than the second probability, the output of the first entity recognition model may be weighted lower than the output of the second entity recognition model. For example, the first probability may be 66.6% and the second probability may be 33.3%, whereby the output of the first entity recognition model may be weighted more than the output of the second entity recognition model by a factor of two if we don't consider any other weighing criteria, including but are not limited to sample size, model performance, and the like.

It should be appreciated that in some embodiments and/or scenarios, probability scores associated with an entity may not add up to 100%, because the scores may be generated by two different models trained using different training datasets. In some embodiments, relative weightings of different model probabilities may be based on a relative quantity and quality of the training data used to train a model, and/or relative performances of the two different models.

As a second example, the first entity recognition model trained to identify instances of the entity “cancer” may label the expression “lung cancer” as “cancer”, with the first probability. A third, multiple entity recognition model trained to identify instances of the entity “cancer” and instances of the entity “anatomy” may label the expression “lung cancer” as “anatomy” with a probability vector including values of 80% (for anatomy) and 10% (for cancer), respectively. As described above, an additional probability score of 10% may be assigned to a third entity “outside”, meaning not cancer or anatomy. As a result of the third, multiple entity recognition model outputting a higher probability of the expression “lung cancer” being an instance of “anatomy” than being an instance of “cancer”, the third, multiple entity recognition model may label “lung cancer” as “anatomy”. To resolve the conflict between the output of the first entity recognition model and the third entity recognition model with respect to labeling “lung cancer”, relative weightings of the two model outputs may be assigned based on the probabilities. The first probability may be compared with the highest probability value of the probability vector (e.g., 80%). If the first probability is higher than the highest probability value of the probability vector, the output of the first entity recognition model may be weighted higher than the output of the third entity recognition model. If the first probability is lower than the highest probability value of the probability vector, the output of the first entity recognition model may be weighted lower than the output of the third entity recognition model.

At 604, method 600 includes adjusting the initial weightings based on relative sizes of labeled data sets used to train each entity recognition model. A first probability outputted by a first entity recognition model may be higher than a second probability outputted by a second entity recognition model. However, an accuracy of the first probability may partially depend on a size (e.g., a quantity of data) of a first labeled dataset used to train the first entity recognition model (e.g., dataset 212), and an accuracy of the second probability may partially depend on a size of a second labeled dataset used to train the second entity recognition model (e.g., dataset 214). The size of the second labeled dataset may be greater than the size of the first dataset. For example, a second entity labeled in the second labeled dataset (e.g., entity 204) may be more commonly found in medical records than a first entity labeled in the first labeled dataset (e.g., entity 204), whereby an amount of text data available for generating the second dataset may be larger than an amount of text data available for generating the first dataset.

As a result of the second dataset being larger than the first dataset, the second probability may be more accurate than the first probability. Therefore, the initial weightings assigned based on the first probability and the second probability may be adjusted to account for a difference between the size of the first labeled dataset and the second labeled dataset. If the first labeled dataset is smaller than the second labeled dataset, the weighting of the first entity recognition model may be reduced and/or the weighting of the second entity recognition model may be increased. If the second labeled dataset is smaller than the first labeled dataset, the weighting of the second entity recognition model may be reduced and/or the weighting of the first entity recognition model may be increased. For example, a weighted probability for the first model may be a*Probability 1, and a weighted probability for the second model could be b*Probability 2, where a & b may be chosen based on criteria related to size of the relevant datasets.

At 606, method 600 includes adjusting the weightings based on a similarity of labeled data sets used to train each entity the mission model to the medical report. A first probability outputted by a first entity recognition model may be higher than a second probability outputted by a second entity recognition model. However, an accuracy of the first probability may partially depend a similarity of a first labeled dataset used to train the first entity recognition model with the medical report, and an accuracy of the second probability may partially depend on a similarity of a second labeled dataset used to train the second entity recognition model with the medical report. The second labeled dataset may be more similar to the medical report than the first labeled dataset, whereby the accuracy of the second probability may be greater than the accuracy of the first probability. Therefore, the initial weightings assigned based on the first probability and the second probability may be adjusted to account for the difference in similarity between the first labeled dataset and the second labeled dataset. If text data used to generate the first labeled dataset is more similar to the medical report than text data used to generate the second labeled dataset, the weighting of the first entity recognition model may be increased and/or the weighting of the second entity recognition model may be decreased. If text data used to generate the first labeled dataset is less similar to the medical report than text data used to generate the second labeled dataset, the weighting of the first entity recognition model may be reduced and/or the weighting of the second entity recognition model may be increased. For example, a weighted probability for the first model may be a*Probability 1, and a weighted probability for the second model could be b*Probability 2, where a and b may be chosen based on the similarity of the medical report with training data of the first model and the second model.

At 608, method 600 includes adjusting the weightings based on a model fusion analysis, in which an output of one or more entity recognition models are compared with an output of a reference multiple entity recognition model trained on entities of the one or more entity recognition models. Adjusting the weightings based on the model fusion analysis is described in greater detail below, reference to FIG. 7.

At 610, method 600 includes assigning a label associated with the model output that has been assigned the highest weighting, and method 600 ends.

FIG. 7 shows an exemplary method 700 for resolving entity labeling conflicts in outputs of a plurality of entity recognition models within a patient summary system (e.g., patient summary system 102), based on a model fusion analysis. In the model fusion analysis, the outputs are compared with a reference output of a multiple entity recognition model to determine a degree of agreement. A relative weighting of the outputs of the plurality of entity recognition models may be adjusted based on each entity recognition model's degree of agreement with the reference output. The relative weightings of the plurality of entity recognition models may be used to determine a most accurate classification of a text expression as an instance of an entity, in a medical report entered as input into each of the plurality of entity recognition models. In various embodiments, method 700 may be executed as part of method 600 described above in reference to FIG. 6.

Method 700 begins at 702, where method 700 includes receiving an expression labeled differently by two or more entity recognition models receiving a same medical report as input. Each of the two or more entity recognition models may be trained to identify instances of a different entity in the medical report. For example, a first entity recognition model of the two or more entity recognition models may be trained to identify instances of “cancer” in the medical report, and a second entity recognition model of the two or more entity recognition models may be trained to identify instances of “treatment” in the medical report. An expression “tumor removal” may be classified as an instance of “cancer” by the first entity recognition model, and classified as an instance of “treatment” by the second entity recognition model, generating a labeling conflict in the medical report.

At 704, method 700 includes entering the medical report into a trained multiple entity recognition model to generate a labeled version of the medical report, where the labeled version includes labeled instances of entities on which the two or more entity recognition models have been trained. For example, if the first entity recognition model in the example above is trained to identify instances of “cancer” in the medical report, and the second entity recognition model is trained to identify instances of “treatment” in the medical report, the medical report may be inputted into a multiple entity recognition model trained to identify instances of both “cancer” and “treatment”.

It should be appreciated that in some scenarios, a text expression of the medical report may be more reliably or accurately identified as an instance of an entity by a multiple entity recognition model trained to recognize two or more entities than an entity recognition model trained to recognize a single entity. A second entity may provide a context for a first entity during training of the multiple entity recognition model that increases an accuracy of its output. For example, the first entity and the second entity may be commonly found in a same sentence of the medical report, whereby an adjacency of the second entity to the first entity may be taken into consideration by the multiple entity recognition model to increase output accuracy.

At 706, method 700 includes extracting a probability vector for the received text expression from an output of multiple entity recognition model. As described above in reference to FIG. 6, the multiple entity recognition model may output a probability vector for each labeled text expression. The probability vector includes various probability values, where each probability value represents a probability of the text expression being correctly identified by one of the entities on which the multiple entity recognition model was trained. For example, if the multiple entity recognition model was trained to identify instances of two entities, a probability vector may be assigned by the multiple entity recognition model to each identified instance of either of the two entities, where the probability vector includes a first probability value indicating a probability of the text expression being an instance of a first entity of the two entities, and a second probability value indicating a probability of the text expression being an instance of a second entity of the two entities.

At 708, method 700 includes labeling the text expression as an instance of the entity having a highest probability in the probability vector. The highest probability may be referred to as a reference probability.

At 710, method 700 includes determining whether the label assigned to the instance matches one or more labels assigned by the two or more entity recognition models (e.g., whether the output of any of the two or more entity recognition models matches the output of the multiple entity recognition model). If at 710 it is determined that the assigned entity label does not match one or more labels assigned by the two or more entity recognition models, method 700 proceeds to 716. At 716, method 700 includes not adjusting the weightings assigned to the two or more entity recognition models, and method 700 ends.

Alternatively, if at 710 it is determined that the label assigned to the instance by the multiple entity recognition model matches one or more labels assigned by the two or more entity recognition models, method 700 proceeds to 712. At 712, method 700 includes comparing the reference probability (e.g., the probability associated with the entity label assigned by the multiple entity recognition model) to probabilities associated with the one or more matching labels assigned by the two or more entity recognition models. The probabilities associated with the one or more matching labels may be outputted by the respective entity recognition models, as described above in reference to FIG. 6.

At 714, method 700 includes determining whether a difference between each probability associated with the one or more matching labels and the reference probability falls within a threshold difference. In some embodiments, the threshold difference may be a fixed number, such as 0.2 (e.g., 20%). In other embodiments, the threshold difference may not be fixed, and may be calculated based on various factors.

If at 714 it is determined that the difference is within the threshold difference, method 700 proceeds to 718. At 718, method 700 includes increasing the weighting of the matching label. In other words, if the output of an entity recognition model matches the output of the reference multiple entity recognition model within the threshold difference, the weighting of the output of the entity recognition model is increased.

Alternatively, if at 714 it is determined that the difference is not within the threshold difference, method 700 proceeds to 716. At 716, method 700 includes not increasing the weighting of the matching label, where the weighting of the matching label may not be adjusted, and method 700 ends.

For example, in one embodiment, the threshold difference may be 0.2. If the reference probability is 0.8, and a probability associated with a matching label outputted by an entity recognition model is 0.65, the difference (e.g., 0.8−0.65=0.15) is within the threshold difference of 0.2, whereby the answer is YES and method 700 proceeds to 718. If the probability associated with the matching label outputted by an entity recognition model is 0.55, the difference (e.g., 0.8−0.55=0.25) is not within the threshold difference of 0.2, whereby the answer is NO and method 700 proceeds to 716.

Thus, methods and systems are provided a patient summary system for summarizing patient information in digitized medical reports, for example, of an EMR of a patient, based on an identification of entities of interest within the digitized medical reports. The entities of interest may be identified and labeled by a plurality of entity recognition models, each of which may be trained to identify a single entity. Outputs of each of the entity recognition models may be aggregated to generate a labeled version of a medical report. The patient summary system may then extract instances of the entities of interest from the medical report, and generate a summary that may be formatted and/or customized for a caregiver. Such extraction may be achieved with more efficient processing by a processor because labeled versions of the report may be more easily assembled into a hierarchical data structure for faster and more efficient searching to thus identify relevant portions of a medical report. The caregiver may specify one or more entities that they are interested in, and the patient summary system may generate a summary specific to those entities. The summary may include, for example, labeled excerpts of the medical report, and/or patient information related to the entities. By viewing the summary rather than reviewing the medical report, the caregiver may save time, allowing the caregiver to find information more quickly. By not having to review a plurality of medical reports in an EMR when seeking patient information, an efficiency of the caregiver and an amount of time the caregiver has to attend to other tasks may be increased. Further, the labeled excerpts may be formatted using labels of differing colors, shading, highlighting, formatting, or other features such that the caregiver may quickly scan for entities of interest, saving the caregiver additional time.

By using separate entity recognition models to identify each entity of interest, and then aggregating the outputs of a plurality of entity recognition models, an accuracy of the entity identification overall may be increased. For example, each entity recognition model may be trained on a different labeled dataset that is curated to maximize a performance of the entity recognition model with respect to the respective entity. Additionally, in some embodiments, one or more of the entity recognition models may be multiple entity recognition models that are trained to identify more than one entity. By comparing outputs of outputs of entity recognition models trained on a single entity with outputs of the multiple entity recognition models, an accuracy of the entity labeling may be increased. For example, in a scenario where a text expression is recognized as two different entities by two different entity recognition models, a multiple entity recognition model trained to recognize both entities may be used to determine a most accurate entity classification.

The technical effect of generating a patient summary of a medical report using separately trained entity recognition models to identify entities of interest in the medical report is that an amount of time spent by a caregiver reviewing patient data may be reduced.

The disclosure also provides support for a method, comprising: receiving text data of a patient, entering the text data as input into a plurality of entity recognition models, each entity recognition model of the plurality of entity recognition models trained to label instances of a respective entity in the text data; aggregating the labeled text data outputted by each entity recognition model; generating a summary of the text data based on the aggregated labeled text data; and displaying and/or saving the summary and/or the aggregated labeled text data. In a first example of the method, the entity recognition models are neural network models. In a second example of the method, optionally including the first example, each entity recognition model of the plurality of entity recognition models is trained on a respective labeled dataset, the respective labeled dataset including a plurality of labeled instances of the respective entity. In a third example of the method, optionally including one or both of the first and second examples, each respective labeled dataset includes instances of entities with a targeted frequency, a targeted length, and a targeted degree of adjacency. In a fourth example of the method, optionally including one or more or each of the first through third examples, an entity recognition model outputs, for each text expression labeled as an entity in the text data, a probability of the text expression being an instance of the respective entity. In a fifth example of the method, optionally including one or more or each of the first through fourth examples, aggregating the labeled text data outputted by each entity recognition model further comprises, for each text expression in the labeled text data that is labeled as an entity by at least two entity recognition models, selecting a most accurate entity label based on relative weightings of outputs of the at least two entity recognition models. In a sixth example of the method, optionally including one or more or each of the first through fifth examples, the weightings are assigned based on the probabilities outputted by the respective entity recognition models of the at least two entity recognition models. In a seventh example of the method, optionally including one or more or each of the first through sixth examples, assigning the weightings further comprises: entering the text data as input into a multiple entity recognition model trained to label instances of a plurality of entities in the text data, for each entity in the labeled text data that is labeled by at least two entity recognition models: comparing a reference label of the entity labeled by the multiple entity recognition model with labels of the entity labeled by the at least two entity recognition models, responsive to a label of the entity generated by an entity recognition model of the at least two entity recognition models matching the reference label within a threshold difference, increasing a weighting of the entity recognition model. In an eighth example of the method, optionally including one or more or each of the first through seventh examples, a weighting of an output of an entity recognition model is adjusted based on a relative similarity of the text data to a labeled dataset used to train the entity recognition model. In a ninth example of the method, optionally including one or more or each of the first through eighth examples, a weighting of an output of an entity recognition model is adjusted based on a size of a labeled dataset used to train the entity recognition model. In a tenth example of the method, optionally including one or more or each of the first through ninth examples, the method further comprises: adjusting or changing a label of the aggregated labeled text data, prior to generating the summary, based on clinical context-based knowledge obtained from one or more domain specific tools. In a eleventh example of the method, optionally including one or more or each of the first through tenth examples, the method further comprises: adjusting or changing a label of the aggregated labeled text data, prior to generating the summary, based on applying one or more grammar-based rules. In a twelfth example of the method, optionally including one or more or each of the first through eleventh examples, the summary includes at least one of: a predicted number of each entity recognized in the text data, examples of the entities recognized in the text data, patient data associated with an entity recognized in the text data, and labeled text data. In a thirteenth example of the method, optionally including one or more or each of the first through twelfth examples, the text data is medical report of the patient stored in an Electronic Medical Record (EMR) of the patient.

The disclosure also provides support for a system, comprising: one or more processors storing executable instructions in non-transitory memory that, when executed, cause the one or more processors to: receive a medical report of a patient from an Electronic Medical Record (EMR) database, enter the medical report as input into a plurality of entity recognition models, each entity recognition model of the plurality of entity recognition models trained to identify instances of a respective entity in the medical report, resolve conflicts between entities identified differently by different entity recognition models, generating a patient summary, the patient summary including information on the instances of the resolved entities identified in the medical report, and displaying the summary on a display device of the system and/or saving the summary in the non-transitory memory. In a first example of the system, resolving the conflicts between the entities identified differently by different entity recognition models further comprises selecting an identified entity of conflicting identified entities by at least one of: comparing probabilities of the conflicting identified entities being accurate, the probabilities outputted by the respective entity recognition models, comparing the probabilities of the conflicting identified entities being accurate with a reference probability of an identified entity being accurate, the reference probability assigned by a multiple entity recognition model trained to identify a plurality of entities in the medical report, comparing a similarity of the medical report to respective labeled datasets used to train the respective entity recognition models, and comparing a relative size of the respective labeled datasets. In a second example of the system, optionally including the first example, prior to generating the summary, the resolved entities are further refined by one of: using domain specific tools to change a first identified entity to a second identified entity based on clinical context-based knowledge, and using natural language processing (NLP) to change a first identified entity to a second identified entity based on grammar-based rules. In a third example of the system, optionally including one or both of the first and second examples, the summary includes at least one of: a number of each entity identified in the medical report, a listing of one or more entities identified in the medical report, patient data related to one or more entities identified in the medical report, and text of the medical report including labeled entities identified in the text.

The disclosure also provides support for a method, comprising: training each entity recognition model of a plurality of entity recognition models on a different dataset, wherein each different dataset includes a plurality of instances of a pre-defined entity, and each instance of the plurality of instances is labeled as being an instance of the pre-defined entity. In a first example of the method, the plurality of instances appear in the dataset with a target frequency, a target length, and a target adjacency.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
receiving text data of a patient;
entering the text data as input into a plurality of entity recognition models, each entity recognition model of the plurality of entity recognition models trained to label instances of a respective entity in the text data;
aggregating the labeled text data outputted by each entity recognition model;
generating a summary of the text data based on the aggregated labeled text data; and
displaying and/or saving the summary and/or the aggregated labeled text data.

2. The method of claim 1, wherein the entity recognition models are machine learning (ML) models.

3. The method of claim 1, wherein each entity recognition model of the plurality of entity recognition models is trained on a respective labeled dataset, the respective labeled dataset including a plurality of labeled instances of the respective entity.

4. The method of claim 3, wherein each respective labeled dataset includes instances of entities with a targeted frequency, a targeted length, and a targeted degree of adjacency.

5. The method of claim 1, wherein an entity recognition model outputs, for each text expression labeled as an entity in the text data, a probability of the text expression being an instance of the respective entity.

6. The method of claim 5, wherein aggregating the labeled text data outputted by each entity recognition model further comprises, for each text expression in the labeled text data that is labeled as an entity by at least two entity recognition models, selecting a most accurate entity label based on relative weightings of outputs of the at least two entity recognition models.

7. The method of claim 6, wherein the weightings are assigned based on the probabilities outputted by the respective entity recognition models of the at least two entity recognition models.

8. The method of claim 7, wherein assigning the weightings further comprises:
entering the text data as input into a multiple entity recognition model trained to label instances of a plurality of entities in the text data;
for each entity in the labeled text data that is labeled by at least two entity recognition models:
comparing a reference label of the entity labeled by the multiple entity recognition model with labels of the entity labeled by the at least two entity recognition models;
responsive to a label of the entity generated by an entity recognition model of the at least two entity recognition models matching the reference label within a threshold difference, increasing a weighting of the entity recognition model.

9. The method of claim 7, wherein a weighting of an output of an entity recognition model is adjusted based on a relative similarity of the text data to a labeled dataset used to train the entity recognition model.

10. The method of claim 7, wherein a weighting of an output of an entity recognition model is adjusted based on a quantity or quality of data of a labeled dataset used to train the entity recognition model.

11. The method of claim 1, further comprising adjusting or changing a label of the aggregated labeled text data, prior to generating the summary, based on clinical context-based knowledge obtained from one or more domain specific tools.

12. The method of claim 1, further comprising adjusting or changing a label of the aggregated labeled text data, prior to generating the summary, based on applying one or more grammar-based rules.

13. The method of claim 1, wherein the summary includes at least one of:
a predicted number of each entity recognized in the text data;
examples of the entities recognized in the text data;
patient data associated with an entity recognized in the text data; and
labeled text data.

14. The method of claim 1, wherein the text data is medical report of the patient stored in an Electronic Medical Record (EMR) of the patient.

15. A system, comprising:
one or more processors storing executable instructions in non-transitory memory that, when executed, cause the one or more processors to:
receive a medical report of a patient from an Electronic Medical Record (EMR) database;
enter the medical report as input into a plurality of entity recognition models, each entity recognition model of the plurality of entity recognition models trained to identify instances of a respective entity in the medical report;
resolve conflicts between entities identified differently by different entity recognition models;
generating a patient summary, the patient summary including information on the instances of the resolved entities identified in the medical report; and
displaying the summary on a display device of the system and/or saving the summary in the non-transitory memory.

16. The system of claim 15, where resolving the conflicts between the entities identified differently by different entity recognition models further comprises selecting an identified entity of conflicting identified entities by at least one of:
comparing probabilities of the conflicting identified entities being accurate, the probabilities outputted by the respective entity recognition models;
comparing the probabilities of the conflicting identified entities being accurate with a reference probability of an identified entity being accurate, the reference probability assigned by a multiple entity recognition model trained to identify a plurality of entities in the medical report;
comparing a similarity of the medical report to respective labeled datasets used to train the respective entity recognition models; and
comparing a relative size of the respective labeled datasets.

17. The system of claim 15, wherein prior to generating the summary, the resolved entities are further refined by one of:
using domain specific tools to change a first identified entity to a second identified entity based on clinical context-based knowledge; and
using natural language processing (NLP) to change a first identified entity to a second identified entity based on grammar-based rules.

18. The system of claim 15, wherein the summary includes at least one of:
a number of each entity identified in the medical report;
a listing of one or more entities identified in the medical report;
patient data related to one or more entities identified in the medical report; and
text of the medical report including labeled entities identified in the text.

19. A method, comprising:
training each entity recognition model of a plurality of entity recognition models on a different dataset, wherein each different dataset includes a plurality of instances of a pre-defined entity, and each instance of the plurality of instances is labeled as being an instance of the pre-defined entity.

20. The method of claim 19, wherein the plurality of instances appear in the dataset with a target frequency, a target length, and a target adjacency.
